(19) United States
(12) Patent Application Publication
(10) Pub. No.: US 2008/0299130 A1
(43) Pub. Date: Dec. 4, 2008

(54) Methods And Compositions For The Treatment Of Ocular Neovascularization

(75) Inventor: Jayakrishna Ambati, Lexington, KY (US)

Correspondence Address: MCDERMOTT WILL & EMERY LLP, 600 13TH STREET, N.W., WASHINGTON, DC, 20005-3096, US

(73) Assignee: UNIVERSITY OF KENTUCKY RESEARCH FOUNDATION, Lexington, KY (US)

(21) Appl. No.: 11/579,487

(22) PCT Filed: May 3, 2005
(86) PCT No.: PCT/US2005/015231

§ 371 Date: Jul. 14, 2008

Related U.S. Application Data
(60) Provisional application No. 60/567,553, filed on May 4, 2004.

(51) Int. Cl. A61K 39/395; A61K 31/713; A61P 27/02

(52) U.S. Cl. 424/158.1; 514/44

(57) ABSTRACT

The invention relates to compositions and methods for the treatment or prevention of ocular neovascularization by reducing macrophage infiltration into the eye. The compositions of the invention include an antagonist of MCP-1 and/or CCR2 that blocks MCP-1 binding to or activation of CCR2y.

### BACKGROUND OF THE INVENTION

Age-related macular degeneration (AMD) is the leading cause of irreversible blindness among the elderly on three continents. Yet little is known about the molecular mechanisms of choroidal neovascularization (CNV), the angiogenic process responsible for the majority of severe vision loss in patients with AMD, as well as patients suffering from other retinopathies, such as diabetic retinopathy or retinopathy of prematurity.

The presence of macrophages in histological studies of CNV has elicited interest in their role in the development of neovascularization associated with retinopathies. The spatiotemporal distribution of macrophages correlates with arborizing CNV in humans and in animal models. In patients with AMD, macrophages are in close proximity to thinned and perforated areas of Bruch's membrane, and participate in digesting the outer collagenous zone of Bruch's membrane, both of which facilitate the subretinal entry of CNV. (van der Schaft et al. (1993) Br. J. Opthalmology 77, 657-661.

There is a need for methods and agents for preventing macrophage infiltration into the diseased or damaged eye, such as an eye afflicted with ocular neovascularization, and in particular choroidal neovascularization.

### SUMMARY OF THE INVENTION

In one aspect of the invention there is provided a composition for treatment or prevention of ocular neovascularization comprising an effective amount of a MCP-1 antagonist and a CCR2 antagonist that blocks MCP-1 binding to or activation of CCR2. In one embodiment, the composition further comprises an anti-inflammatory agent and/or steroid drug. In another embodiment, the composition further comprises a VEGF antagonist.

In another aspect of the invention there is provided a method for treating or preventing ocular neovascularization of a subject comprising administering to a subject an effective amount of a MCP-1 antagonist or a CCR2 antagonist, or a combination thereof. Preferred MCP-1 antagonists and CCR2 antagonists include small molecules, peptides, nucleic acids in either sense or anti-sense orientation, either single or double stranded nucleic acids specifically targeted to MCP-1 or CCR2, and most preferably, anti-MCP-1 or anti-CCR2 antibodies and functional antibody fragments thereof. In one embodiment, the MCP-1 and/or CCR2 antagonist is administered systemically. In another embodiment, the MCP-1 and/or CCR2 antagonist is administered topically or via direct injection into the eye. In yet another embodiment, the method further includes co-administration of an effective amount of a VEGF antagonist. The compositions and methods of the invention are particularly useful for the treatment of choroidal neovascularization such as that associated with age related macular degeneration.

MCP-1 is also referred to as Ccl-2 in the scientific literature. Thus, reference to MCP-1 and MCP-1^{-/-} mice herein is a direct reference to Ccl-2 and Ccl-2^{-/-} mice, respectively.

### BRIEF DESCRIPTION OF THE DRAWINGS

FIG. 1. MCP-1 expression and macrophage infiltration is localized in area of laser injury. (A) Choroidal base of laser lesion 2 days after injury in a wild-type mouse shows no MCP-1 immunostaining in the choroidal endothelium (stained green by FITC-conjugated Isolectin B4) distant from the central lesion. MCP-1 staining is abundant at the edge (red) and the center of the laser scar (yellow color results from digital merging of red and green). (B) 3 days after laser injury in a wild-type mouse, macrophages stained by Cy5-conjugated F4/80 (blue) colocalize with endothelial cells (green) in the middle of the CNV lesion. Colocalization by merging yields a cyan color. 1 μm sections are shown. (C) MCP-1 protein expression, normalized to total protein and maximal MCP-1 response, peaks at day 2 after laser injury. N=4 per group. (D) Peak VEGF protein expression, normalized to total protein, occurs on day 3 (data not shown) and is reduced in MCP-1^{-/-} and CCR2^{-/-} mice compared with controls. N=4 per group. Scale bars, 100 μm (A) and 50 μm (B).

FIG. 2. Macrophage entry into CNV during the first week after laser injury. (A) One day after laser injury in a wild-type animal, CCR2 staining (red) is concentrated at the choroidal base of the scar (digital merging with FITC-Isolectin B4 stained endothelial cells yields yellow color) and the adjacent choroid. (B) 3 days after laser injury in a wild-type animal, CCR2 staining is restricted to the area of the laser scar (yellow—arrows) and no staining is visible in the adjacent choroid (green). (C) 5 days after laser injury in a wild-type animal, minimal, CCR2 staining is evident (arrow). (D) 1 day after laser injury in a wild-type animal, dual immunostaining with CCR2 (red) and F4/80 (blue) demonstrates marked spatial colocalization (arrows). Digital merge yields purple. (E) 3 days after laser injury in a wild-type animal, F4/80 staining (blue) is abundant but comparatively less of the laser scar stains positive for CCR2 (red). 1 μn sections are shown. Scale bar, 100 μm. (F) Number of F4/80 (squares) and CCR2 (circles) positive cells in the choroid detected by flow cytometry prior to laser injury (day 0) and on the first 5 days after injury. Values normalized to maximal F4/80 response. N=4 per group. (G) Microglia (arrows) stained by 5D4 (red or yellow when merged with green lectin staining) are identified in the neural retina in the layers beneath the retinal vessels (stained green by FITC-Isolectin B4) but not near the CNV lesion (perimeter outlined in white) or within it (H).

FIG. 3. CNV volume is markedly diminished in MCP-1^{-/-} and CCR2^{-/-} mice two weeks after laser injury. Stacked confocal images of tissue labeled with FITC-isolectin B4 (A-D) or CD31 (E-H) within laser scars show CNV membranes with significantly smaller volumes in MCP-1^{-/-}(A) and CCR2^{-/-} mice (B) compared to wild-type (C) or CCR2^{+/+} mice (D). Scale bar, 100 μm (A-D), 50 μm (E-H). The volume per lesion (mean ±s.e.m.) in MCP-1^{-/-} or CCR2^{-/-} mice was significantly less than in wild-type C57BL/6J or CCR2^{+/+} mice, measured by CD31 (black bars) or FITC-Isolectin B4 (white bars) staining. *P<0.0001. N 7 animals for all groups.

FIG. 4. Angiographic leakage of laser-induced CNV is diminished in MCP-1^{-/-} and MCP-1^{-/-} mice. (A) Significantly fewer CNV lesions in MCP-1^{-/-} and CCR2^{-/-} mice show pathologically significant leakage (grade 2B) compared to control mice. *P<0.05. Representative late phase (6-8 min) fluorescein angiograms 2 weeks after laser injury of MCP-1^{-/-} (B) and MCP-1^{-/-} mice (C) show markedly diminished leakage compared to laser lesions in wild-type mice (D). Early phase angiograms (1-2 min) showed minimal hyperfluorescence in regions that leaked late.

FIG. 5 is a bar graph demonstrating that recombinant mouse MCP-1 restores CNV in PBS-treated MCP-1^{-/-} mice in a manner insensitive to anti-VEGF-A antibody (2 ng). *P<0.05 compared with PBS. Control=wild-type mouse; all other data was obtained using MCP-1 knock out mice.

FIG. 6 is a bar graph demonstrating that recombinant mouse VEGF-A164 does not increase CNV in PBS-treated MCP-1 KNOCK OUT mice. *P<0.05 compared with PBS. Control=wild-type mouse; all other data obtained using MCP-1 knock out mice.

FIG. 7 is a bar graph showing the effect of an anti-MCP-1 antibody (1 ng) on choroidal macrophage recruitment in PBS-treated MCP-1^{-/-} mice. The four bars to the left of FIG. 5 show data obtained with treated wild-type mice; the fifth and sixth bars from the left show data obtained using MCP-1^{-/-} mice; and the two remaining bars at the right of the figure show data obtained with Vegfr1 tk^{-/-} mice. *P<0.5 compared with PBS-treated mice of the same genetic strain. #P<0.5 compared with PBS-treated wild-type mice.

### DETAILED DESCRIPTION OF THE INVENTION

Age-related macular degeneration (AMD) is a leading cause of vision loss worldwide. Laser injury induced choroidal neovascularization (CNV) is an experimental model of the exudative stage of AMD, the leading cause of blindness in patients with AMD. The specific mechanisms of macrophage recruitment in this injury model are described herein, and CNV responses in mice genetically deficient in genes coding for monocyte chemoattractant protein-1 (MCP-1) or macrophage inflammatory protein-1α (CCL3) or their cognate receptors, CCR2 or CCR5 are analyzed. The results show that MCP-1 and CCL3 mRNA synthesis is upregulated soon after laser injury. Compared to wild-type mice, the number of macrophages recruited to the choroid following laser injury is markedly reduced in each knockout strain. The expression of vascular endothelial growth factor (VEGF) following laser injury and preceding CNV development is similarly diminished in knockout mice compared with wild-type mice. The CNV response is markedly diminished in each of these knockout strains compared with wild-type mice, and correlates with the number of recruited macrophages and VEGF levels. These findings define the role of the macrophage as a critical component in initiating the laser-induced CNV response.

It is demonstrated herein that MCP-1^{-/-}, CCR2^{-/-}, CCL3^{-/-}, and CCR5^{-/-} mice develop smaller CNV membranes than wild-type mice. These data are consistent with MCP-1 expression in CNV membranes in patients with AMD (Spandau et al., (2000), Invest Opthalmol Vis Sci 41, S836; Grossniklaus, et al., (2002), Mol Vis 8, 119-126) and suppression of laser-induced CNV in mice selectively depleted of macrophages (Sakurai et al., IVOCS, 44(8): 3578-3585)). Collectively these data implicate macrophages, which previous histopathological studies of experimental and clinical CNV have shown to be in close association with new vessels, as playing a causal not a coincidental role in the development of neovascularization in the eye. In particular, recruitment of blood-borne monocytes and macrophages appears to play the major role in initiating the angiogenic response, perhaps via delivery of VEGF, the putative angiogen that drives CNV. Although a potential late role for resident macrophages/microglia is not excluded in this model, the facts that macrophage recruitment is completed within the first few days after laser injury and that the maximal VEGF response, which occurs at day three, is correlated with the influx of recruited macrophages and with the final CNV volume all suggest that recruited, and not resident, macrophages play the major role.

It is shown herein that the inhibition of CNV, although marked, was incomplete in both knockout groups, suggesting the presence of alternate mechanisms of angiogenesis. Nevertheless, the substantial inhibition of CNV demonstrates that the MCP-1-CCR2 and CCL3-CCR5 axes play key roles in initiating or at least facilitating CNV, particularly because their expression is abundant in the site of CNV early after laser injury. Genetic ablation of these chemokines or their receptors, while markedly inhibiting CNV, did not match the near abolition induced by pharmacological depletion of macrophages by clodronate liposome treatment (Sukarai et al. (2003)). This probably is due to the fact that the complete contribution of monocytes to CNV induction may not have been extracted in this paradigm of individually disrupting these molecules because these two axes of macrophage trafficking likely act synergistically. In other sites of ocular neovascularization such as the cornea, CCR2 and CCR5 appear to differentially mobilize pro-angiogenic macrophages (Ambati, et al., (2003), Invest Opthalmol Vis Sci 44, 590-593, Ambati, et al., (2003), Cornea 22, 465-467).

CCR2 expression, originally described only on monocytes, recently has been demonstrated on human microvascular and umbilical vein endothelial cells, albeit at much lower levels (Weber, et al., (1999), Arterioscler Thromb Vasc Biol 19, 2085-2093; Salcedo, et al., (2000), Blood 96, 34-40). Moreover, in C57BL/6J mice CCR2 expression among hematopoietic cells is almost entirely restricted to monocytes (Mack, et al., (2001), J Immunol 166, 4697-4704). It is shown herein that CCR2 staining in the laser lesion parallels that of the pan-macrophage marker F4/80, both spatially and temporally. In contrast, endothelial cell staining increases during the decline of CCR2 staining after day three. Together these data imply that the predominant source of CCR2 expression emanates from monocytes. MCP-1 plays an important role in angiogenesis, indirectly by recruiting macrophages, which can secrete VEGF and matrix metalloproteinases and directly by inducing endothelial cell migration and proliferation (Weber, et al., (1999), Arterioscler Thromb Vasc Biol 19, 2085-2093; Salcedo, et. al., (2000), Blood 96, 34-40). However, it has been shown previously that specific macrophage depletion reduces CNV volume by 90% despite the secretion of MCP-1 and CCL3 (Sakurai, et al., (2003) Invest Opthalmol Vis Sci 44, 3578-3585). Therefore, the primary pro-angiogenic action of MCP-1 in CNV seems to be due to its effect on monocyte recruitment, although a minor effect on endothelial cells cannot be excluded.

In addition to recruiting monocytes, MCP-1 promotes monocyte adherence to the endothelium under flow conditions. This may represent another mechanism that mediates the pro-angiogenic effect of MCP-1 as previously it was shown that the leukocyte adhesion molecules CD18 and ICAM-1 play a key role in laser-induced CNV (Sakurai, et al., (2003,) Invest Opthalmol Vis Sci 44, 2743-2749).

These findings are relevant to neovascularization observed in various retinopathies and eye pathologies, including AMD, CNV, diabetic retinopathy, and retinopathy of prematurity. For example, although the laser injury model may involve processes not relevant to AMD, it captures many of the important features of the human condition. Laser photocoagulation that disrupts Bruch's membrane can induce CNV in humans (Francois, et al., (1975), Am J Opthalmol 79, 206-210). Both in experimental models and in AMD newly formed vessels that are functionally incompetent project into the subretinal space through defects in Bruch's membrane. Aggregation of leukocytes near arborizing neovascular tufts is another shared feature of experimental and clinical CNV. Immunostaining has demonstrated the presence of VEGF and its receptors, basic fibroblast growth factor, transforming growth factor-α, tumor necrosis factor-α, Fas, and Fas-ligand in cells comprising the CNV membranes in both conditions. The laser injury model also has been predictive of therapeutic response in human clinical trials (The Eyetech Study Group (2002), Retina 22, 143-152).

It has been demonstrated that MCP-1^{-/-} or CCR2^{-/-} mice, but not CCL3^{-/-} or CCR5^{-/-} mice spontaneously develop features of AMD, including CNV, as they age (Ambati, et al., (2003), Nat Med 9, 1390-1397). The mechanisms at work in this chronic model of CNV differ from the pro-angiogenic role for MCP-1 and CCR2 in this acute injury model of CNV because CNV in the former model results from the inability to recruit scavenger macrophages needed to clear deposits that accumulate with age, whereas CNV in the latter arises from pro-angiogenic activity of inflammatory macrophages. The similarities and differences in ocular phenotype between mice with deficiencies in MCP-1/CCR2 versus CCL3/CCR5 highlights the functional convergence and divergence in these pathways in acute versus chronic settings.

The development of CNV is inhibited in mice deficient in the MCP-1 or CCR2 gene. This observation suggests that macrophage mobilization by this chemokine-receptor duo is non-redundant in the development of CNV, which is the principal cause of vision loss in AMD, and other retinopathies. These findings may be relevant to neovascularization elsewhere in the body, where leukocytes and growing vessels are often found in close proximity. Because leukocytes are the principal purveyors of host immune defenses, targeted methods of inhibiting MCP-1 or CCR2 function using local drug delivery are desirable.

The present invention provides methods of treatment that target MCP-1 or CCR2, or both, in the treatment and/or prevention of neovascularization associated with various eye pathologies, such as for example, AMD. The invention also provides compositions for topical application to the eye for treatment or prevention of eye pathologies involving neovascularization.

As demonstrated herein, MCP-1/CCR2 play a key role as a causative agent in ocular neovascularization, such as CNV associated with eye pathologies. It is therefore desirable to develop drugs and treatments that can effectively inhibit the ability of MCP-1 to bind to and activate its receptor, CCR2. By interfering with the ability of MCP-1 to activate CCR2, the methods and compositions of the invention inhibit the migration of circulating macrophages to the eye and thereby prevent or inhibit neovascularization.

Candidate drugs for use in the methods and compositions of the present invention include pharmaceutical compounds, small molecules, peptides, proteins, e.g., peptides or proteins that block MCP-1 or CCR2 using KDEL or KDEL-like retention motifs, aptamers, e.g., RNA/DNA aptamer, ribozyme, antibodies, functional antibody fragments (e.g., F(AB) fragments) specific for MCP-1 or CCR-2 and which interfere with MCP-1 binding to or activation of CCR-2, respectively, and nucleic acids, including oligonucleotides and polynucleotides in sense or antisense orientation, and single or double stranded nucleic acid molecules (e.g., siRNA) that target MCP-1 sequences and interfere with MCP-1 gene expression or that target CCR2 and interfere with CCR2 gene expression. Exemplary compounds that inhibit MCP-1 binding to its receptor, CCR2, include those disclosed in U.S. Pat. Nos. 6,653,345; 6,677,365; 6,670,364; and 6,534,521, for example. Exemplary compounds that target CCR2 and inhibit MCP-1 binding and/or activation thereof include the ligands disclosed in J. Med. Chem., 2003, 46:4070-4086. Preferred compounds that either neutralize MCP-1 activity or otherwise inhibit MCP-1 binding to CCR2 include monoclonal antibodies and functional fragments thereof.

As used herein, the term “antibody” refers to an immunoglobulin molecule with a specific amino acid sequence evoked by an antigen, e.g. MCP-1 or CCR2, and characterized by reacting specifically with the antigen in some demonstrable way. The term “antibody” encompasses polyclonal and monoclonal antibody preparations, CDR-grafted antibody preparations, as well as preparations including hybrid antibodies, altered antibodies, F(AB)′.sub.2 fragments, F(AB) molecules, Fv fragments, single domain antibodies, chimeric antibodies and functional fragments thereof which exhibit immunological binding properties of the parent antibody molecule. The antibodies can also be humanized.

The term “monoclonal antibody” is not limited to antibodies produced through hybridoma technology. The term “monoclonal antibody” refers to an antibody or functional fragment thereof that is derived from a single clone, including any eukaryotic, prokaryotic, or phage clone, and not the method by which it is produced.

As used herein, “compound” refers to any agent, chemical substance, or substrate, whether organic or inorganic, or any protein including antibodies and functional fragments thereof, peptides, polypeptides, peptoids, and the like.

As used herein, the term “carrier” refers to a diluent, adjuvant, excipient, or vehicle with which the compositions of the present invention are administered. The term “pharmaceutically acceptable carrier” refers to a carrier that may be administered to a subject, and which does not destroy the pharmacological activity thereof and is nontoxic when administered in doses sufficient to deliver a therapeutic amount of the compound. In a first aspect of the invention, there is provided a method for screening candidate drugs for the treatment or prevention of neovascularization in the eye. In this aspect of the invention a test animal, such as a mouse, rat, rabbit, monkey, pig, etc. which has undergone laser photocoagulation of at least one eye to provide injury to the Bruch's membrane is provided. The candidate drug is administered to the laser treated eye(s) at various times after treatment, preferably within one day to seven days after treatment, more preferably within one to three days after treatment and most preferably on the second day after treatment. The eye is monitored for the appearance or diminution of neovascularization if neovascularization has already begun at the time the test drug is administered. In one embodiment, both eyes are laser photocoagulated and the test compound is administered to only one eye, thereby allowing direct comparison of the effect of the test drug versus no treatment.

The candidate drug can be pre-screened for its ability to bind to MCP-1 or CCR2 and block MCP-1/CCR2 interaction, neutralize MCP-1 activation of CCR2, inhibit MCP-1 monocyte chemotaxis or mobilize calcium, or otherwise interfere with MCP-1 activation of its receptor, CCR2. Alternatively, test compounds can first be screened in the animal model and those compounds that exhibit an inhibitory effect on neovascularization can then be further screened to determine their effect on MCP-1 and/or CCR2.

The test compound may be administered to the test animal systemically, intravitreously (e.g., by injection or sustained delivery implant), transsclerally or topically, and preferably by topical application to the affected eye(s) of the animal. Treated animals are periodically examined to determine the effect of the candidate drug on angiogenesis. A decrease in number of macrophages or a decrease of neovascularization in the treated eye, for example, is an indication of the ability of the candidate drug to effectively treat neovascularization associated with eye pathologies.

Compounds that demonstrate an inhibitory effect on macrophage infiltration to the eye or neovascularization of the injured eye can be further tested to determine their effect on MCP-1 and/or CCR2 by any assay for MCP-1 activity or CCR2 activation, e.g., MCP-1 ability to act as a macrophage chemoattractant or ability to bind MCP-1, respectively.

In a preferred embodiment of this aspect of the invention the test compound is an antibody or functional antibody fragment, most preferably a humanized antibody or functional fragment thereof. Antibodies can be developed by known methods in the art against the MCP-1 protein or CCR-2 protein. The antibodies may be polyclonal antibodies or monoclonal antibodies.

Polyclonal antibodies to MCP-1 or CCR2 can be produced by various procedures well known in the art. For example, purified MCP-1 or CCR2, or both, preferably human MCP-1 and/or human CCR2, can be administered to various host animals including, but not limited to, rabbits, mice, rats, etc. to induce the production of sera containing polyclonal antibodies specific for the antigen. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Such adjuvants are also well known in the art.

Monoclonal antibodies to MCP-1 or CCR2 can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981) (said references incorporated by reference in their entireties).

In another embodiment, MCP-1 activity and/or CCR2/MCP-1 interaction in the eye is inhibited with MCP-1 blocking peptides that bind specifically to and inhibit the active site of MCP-1 or CCR2 blocking peptides that inhibit or interfere with MCP-1 binding and/or activation of CCR2. Human MCP-1 is secreted as a 76 amino acid protein. Chemical synthesis of MCP-1 analogues has revealed that the amino-terminal residues 1-6 are important for receptor recognition and signaling, and modification or removal of the amino terminal region can completely inactive these chemokines (Proost et al., J. Immun. 160:4034-41, (1998), incorporated herein). Examples of amino-terminal truncated versions of MCP-1 useful in the practice of this invention include the following MCP-1 blocking peptides: (MCP-1 residues 7-76), (MCP-1 residues 8-76), (MCP-1 residues 9-76), and an MCP-1 truncation lacking amino acid residues 2-8 (and including residues 1 and 9-76). In one embodiment of this aspect of the invention, the MCP-1 antagonist is MCP-1 lacking amino acids 2-8.

Other examples of blocking peptides useful in the practice of this invention include any peptides that block the activity of MCP-1, including for example, derivatives of MCP-1 such as amino terminal deletions of MCP-1. Studies have shown that amino-terminal truncations of MCP-1, such as, for example, an MCP-1 truncation (including amino acid residues 6-76 of MCP-1) can completely block the chemotactic effect of MCP-1 on monocytes (Proost, supra). Other examples of useful peptide antagonists include MCP-1 fusion peptides, amino terminal modifications of MCP-1 such as N-terminal methylation, amino acid substitutions, glycosylation, proteolytic cleavage, and linkage to an antibody molecule or other cellular ligand.

The MCP-1 blocking peptides of the invention can be produced by chemical synthesis in accordance with art recognized methods and also by incorporating a nucleic acid molecule, encoding the blocking peptide into an expression vector, introducing the expression vector into a host cell and expressing the nucleic acid molecule to yield polypeptide. The polypeptide can then be recovered and purified by any applicable purification method, including ammonium sulfate or ethanol precipitation, acid extraction, anion or cation exchange chromatography, gel filtration, hydrophobic interaction chromatography, affinity chromatography, hydroxyapatite chromatography, and high performance liquid chromatography (“HPLC”).

MCP-1 blocking peptides can also be produced in vivo, for example by delivering a vector containing a DNA molecule encoding a MCP-1 blocking peptide operationally linked to an expression cassette to the eye, in accordance with the methods of the invention described herein.

Compounds that interfere with CCR2 binding of MCP-1 are also useful in the practice of the invention. Anti-CCR2 antibodies, such as the humanized CCR2 antibodies of U.S. Pat. No. 6,696,550, and U.S. Pat. No. 6,084,075 are useful in the practice of the present invention.

The compounds of the invention may also be combined with one or more additional agents having utility in the treatment of ophthalmic disorders, such as an anti-VEGF antibody or other VEGF antagonist as is known in the art.

### Therapeutic Treatment

The present invention is further directed to methods for preventing and treating neovascularization associated with eye pathologies, in particular, wet AMD, CNV, retinopathy of prematurity, traumatic eye injury, and the like.

Methods of treatment and/or prevention of the present invention comprise administering to a subject in need thereof a MCP-1-inhibitory agent or CCR2 inhibitory agent. For example, the MCP-1 inhibitor and/or CCR2 inhibitor may be administered to a patient, preferably a mammal, most preferably a human, suffering from traumatic eye injury, age related macular degeneration, retinopathy, including retinopathy of prematurity, choroidal neovascularization, or neovascularization associated with any pathological condition of the eye.

The MCP-1- and/or CCR2 inhibitory agent may be administered with a pharmaceutically acceptable carrier. Such pharmaceutical carriers can be sterile liquids, such as water and oils, including those of petroleum, animal, vegetable or synthetic origin, such as peanut oil, soybean oil, mineral oil, and the like. Saline solutions and aqueous dextrose, polyethylene glycol (PEG) and glycerol solutions can also be employed as liquid carriers, particularly for injectable solutions. Suitable pharmaceutical excipients include starch, glucose, lactose, sucrose, gelatin, malt, rice, sodium stearate, glycerol monostearate, glycerol, propylene, glycol, water, and the like. The composition, if desired, can also contain minor amounts of wetting or emulsifying agents, or pH buffering agents. The MCP-1 and/or CCR2 inhibitor or other active agents of the composition may be encased in polymers or fibrin glues to provide controlled release of the active agents. These compositions can take the form of solutions, suspensions, emulsion, tablets, pills, capsules, powders, sustained-release formulations and the like. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the patient. The formulation should suit the mode of administration.

In a preferred embodiment, the composition is formulated in accordance with routine procedures as a pharmaceutical composition adapted for injection into the eye Typically, compositions for injection are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

Compositions of the invention may be administered to the affected eye(s) of a subject by transscleral delivery for example by passive diffusion, controlled release device with or without a remote on-demand delivery system, osmotic pump, or via an implant in the eye, preferably a sustained release implant in the posterior of the eye.

In another preferred embodiment, the composition is administered by topical application to the eye. The compositions are typically administered to the affected eye by applying one to four drops of a sterile solution or suspension, or a comparable amount of an ointment, gel or other solid or semisolid composition, to the surface of the affected eye one to four times per day. However, the compositions may also be formulated as irrigating solutions that are applied to the affected eye during surgical procedures.

Alternatively, the compositions of the invention may be administered systemically.

The ophthalmic compositions of the present invention will contain one or more MCP-1 inhibitors, one or more CCR2 inhibitors, one or more anti-inflammatory agents, or combinations thereof in pharmaceutically acceptable vehicles. Preferably, the compositions of the invention contain one or more MCP-1 inhibitory agents in combination with one or more CCR-2 inhibitory agents. A preferred composition contains an anti-MCP-1-specific antibody or functional fragment thereof that inhibits MCP-1 interactions with CCR-2 and a CCR-2-specific antibody or active fragment thereof that inhibits activation of CCR-2.

The ophthalmic compositions of the present invention may contain one or more MCP-1 inhibitors and/or CCR2 inhibitors in combination with one or a combination of other treatment agents, such as steroid drugs, such as triamcinolone, fluocinolone, anacortave acetate, dexamethasone and combinations thereof; and/or a non-steroidal anti-inflammatory drug, such as celecoxib, VIOXX, flurbiprofen, and aspirin, and combinations thereof. A preferred composition contains both a MCP-1 inhibitory agent and a CCR-2 inhibitory agent, preferably an antibody or functional antibody fragment specific for MCP-1 and CCR-2, respectively, in combination with an anti-inflammatory agent or steroid drug. Additional preferred compositions also contain one or more VEGF inhibitors, such as an anti-VEGF antibody.

Topical compositions will typically have a pH in the range of 4.5 to 8.0. The ophthalmic compositions must also be formulated to have osmotic values that are compatible with the aqueous humor of the eye and ophthalmic tissues. Such osmotic values will generally be in the range of from about 200 to about 400 milliosmoles per kilogram of water (“mOsm/kg”), but will preferably be about 300 mOsm/kg.

Ophthalmic pharmaceutical products are typically packaged in multidose form. Preservatives are thus included to prevent microbial contamination during use. Suitable preservatives include: polyquaternium-1, benzalkonium chloride, thimerosal, chlorobutanol, methyl paraben, propyl paraben, phenylethyl alcohol, edetate disodium, sorbic acid, or other agents known to those skilled in the art. The use of polyquaternium-1 as the antimicrobial preservative is preferred. Typically such preservatives are employed at a level of from 0.001% to 1.0% by weight.

The solubility of the components of the present compositions may be enhanced by a surfactant or other appropriate co-solvent in the composition. Such co-solvents include polysorbate 20, 60, and 80, polyoxyethylene/polyoxypropylene surfactants (e.g., Pluronic F-68, F-84 and P-103), cyclodextrin, or other agents known to those skilled in the art. Typically such co-solvents are employed at a level of from 0.01% to 2% by weight.

The use of viscosity enhancing agents to provide the topical compositions of the invention with viscosities greater than the viscosity of simple aqueous solutions may be desirable to increase ocular absorption of the active compounds by the target tissues or increase the retention time in the eye. Such viscosity building agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose or other agents know to those skilled in the art. Such agents are typically employed at a level of from 0.01% to 2% by weight.

The MCP-1 and/or CCR2 inhibitory compound-containing compositions of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The amount of the active agents of the compositions of the invention which will be effective in the treatment, inhibition and/or prevention of neovascularization of the eye can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses may be extrapolated from dose-response curves derived from in vitro or animal model test systems.

Various delivery systems are known and can be used to administer a composition of the invention, e.g., encapsulation in liposomes, microparticles, microcapsules, recombinant cells capable of expressing the compound, receptor-mediated endocytosis (See, e.g., Wu and Wu, J. Biol. Chem. 262:4429-4432 (1987)), construction of a nucleic acid as part of a retroviral or other vector, etc. The compounds or compositions may be administered together with other biologically active agents, such as an anti-inflammatory agent or steroid drug. Administration is preferably local, either on the surface of the affected eye(s) or injected into the affected eye(s).

Local administration to the affected eye(s) may be achieved by, for example, and not by way of limitation, local infusion during surgery, topical application, e.g., in conjunction with a wound dressing after surgery or via drops or application of a gel or other topical solution, by injection, or by means of an implant, said implant being of a porous, non-porous, or gelatinous material, including membranes, such as sialastic membranes, or fibers. Preferably, when administering a protein, including an antibody or functional fragment thereof, of the invention, care must be taken to use materials to which the protein does not absorb.

In another embodiment, the compound or composition can be delivered in a vesicle, in particular a liposome (See Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.)

In yet another embodiment, the compound or composition can be delivered in a controlled release system. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J., Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the eye.

The following examples are presented for the illustrative purposes and it is to be understood that the present invention is not limited to those precise embodiments, and that various changes and modifications can be effected therein by one skilled in the art without departing from the scope and spirit of the invention as defined by the appended claims.

### EXAMPLES

### Methods

All animal experiments were in accordance with the guidelines of the University of Kentucky IACUC and the Association for Research in Vision and Opthalmology. Male MCP-1^{-/-} and CCR2^{-/-} mice generated as described previously ((Lu, et al., (1998), J Exp Med 187, 601-608; Kuziel, et al., (1997), Proc Natl Acad Sci USA 94, 12053-12058), and wild-type C57BL/6J mice (Jackson Labs, Bar Harbor, Me.) between 6 and 8 weeks of age were used to minimize variability, as age and sex (Tanemura M, et al. IOVS 2001; 42:ARVO Abstract 530) can influence susceptibility to CNV. The knockouts used in the present study represent the 10^{th} backcross generation to C57BL/6J. The CCR2^{-/-} strain was backcrossed to C57BL/6J to generate CCR2^{+/+} mice as a second control group. Genotypes were confirmed by genomic PCR strategies. For all procedures, anesthesia was achieved by intramuscular injection of 50 mg/kg ketamine hydrochloride (Ft. Dodge Animal Health, Fort Dodge, Iowa) and 10 mg/kg xylazine (Phoenix Scientific, St. Joseph, Mo.), and pupils were dilated with topical 1% tropicamide (Alcon, Ft. Worth, Tex.).

### Induction of CNV

Laser photocoagulation (532 nm, 200 mW, 100 ms, 75 μm; OcuLight GL, Iridex, Mountain View, Calif.) was performed on both eyes of each animal by a single individual masked to genetic identity. Laser spots (volume studies: 3/eye; angiography: 4/eye; protein analyses/flow cytometry: 12/eye) were applied in a standardized fashion around the optic nerve, using a slit lamp delivery system and a cover slip as a contact lens. The morphological endpoint of the laser injury was the appearance of a cavitation bubble, a sign thought to correlate with the disruption of Bruch's membrane.

### Volume of CNV

Two weeks after laser injury, eyes were enucleated and fixed with 4% paraformaldehyde (PFA) for 30 minutes at 4° C. Eyecups obtained by removing anterior segments were washed three times in PBS, followed by dehydration and rehydration through a methanol series. After blocking twice with buffer (PBS containing 1% bovine serum albumin (BSA, Sigma-Aldrich, St. Louis, Mo.) and 0.5% Triton X-100 (Sigma-Aldrich)) for 30 minutes at room temperature, eyecups were incubated overnight at 4° C. with 0.5% FITC-Griffonia simplicifolia Isolectin B4 (Vector Laboratories, Burlingame, Calif.) or 0.5% FITC-CD31 (PECAM-1; BD Pharmingen, San Diego, Calif.) (diluted with PBS containing 0.2% BSA and 0.1% Triton X-100), both of which label endothelial cells. After two washings with PBS containing 0.1% Triton X-100, the neurosensory retina was gently detached and severed from the optic nerve. Four relaxing radial incisions were made and the remaining RPE-choroid-sciera complex was flat mounted in anti-fade medium (Immu-Mount Vectashield Mounting Medium, Vector Laboratories) and coverslipped.

Flat mounts were examined with a scanning laser confocal microscope (TCS SP, Leica, Heidelberg, Germany). Vessels were visualized by exciting with blue argon laser wavelength (488 nm) and capturing emission between 515 to 545 nm. Horizontal optical sections (1 μm step) were obtained from the surface of the RPE-choroid-sclera complex. The deepest focal plane in which the surrounding choroidal vascular network connecting to the lesion could be identified was judged to be the floor of the lesion. Any vessel in the laser treated area and superficial to this reference plane was judged as CNV. Images of each section were digitally stored. The area of CNV-related fluorescence was measured by computerized image analysis with the microscope software (TCS SP; Leica). The summation of whole fluorescent area in each horizontal section was used as an index for the volume of CNV. Imaging was performed by an operator masked to genetic identity.

### Immunostaining

At various times during the first week after laser injury, eyes were enucleated under deep anesthesia 30 minutes after they were injected with 1 mL FITC-Griffonia simplicifolia Isolectin B4 via tail vein, and fixed with 4% PFA for 30 minutes at 4° C. Eyecups incubated in 4% PFA were microwaved for 30 seconds, incubated in 4% PFA for 30 minutes on ice, washed three times in PBS, followed by dehydration and rehydration through a methanol series. After blocking twice with blocking buffer for 30 minutes at room temperature, eyecups were incubated overnight at 4° C. with 7 μg/mL of rat anti-mouse MCP-1 (HyCult Biotechnology, Uden, The Netherlands), goat anti-mouse CCR2 (Santa Cruz Biotechnology, San Diego, Calif.), or anti-keratan sulfate proteoglycan (5D4, 1:300; Sekagaku, Tokyo, Japan; gift of J. Wayne Streilein, Harvard Medical School, Boston, Mass.) to label retinal microglia. Following rinses with 0.1% Triton X-100/PBS, tissues were incubated with for 30 minutes at room temperature with 7 μg/mL Texas Red-conjugated secondary antibodies (Santa Cruz Biotechnology). Alternatively, direct immunofluorescence was used to identify macrophages by incubation with 7 μg/mL of Cy5-conjugated F4/80 Ab (Serotec, Oxford, U.K.) for 6 hours at 4° C. RPE-choroid-scleral flat mounts were prepared with or without the neurosensory retina in antifade medium (Immu-Mount; Vector Laboratories) and coverslipped. Image analysis was performed by an operator masked to treatment group assignment.

### VEGF ELISA

The RPE/choroid complex was sonicated in lysis buffer (20 mM imidazole HCl, 10 mM KCl, 1 mM MgCl₂, 10 mM EGTA, 1% Triton X-100, 10 mM NaF, 1 mM Na molybdate, 1 mM EDTA with protease inhibitor (Sigma-Aldrich)) on ice for 15 minutes. VEGF protein levels in the supernatant were determined by ELISA kits (R&D Systems, Minneapolis, Minn.; thresholds of detection 3 pg/mL), at 450/570 nm (Emax, Molecular Devices), and normalized to total protein (Bio-Rad, Hercules, Calif.). Measurements were performed in masked fashion.

### Flow Cytometry

Single cell suspensions isolated from mouse RPE/choroids via collagenase D (20 U/mL; Roche Diagnostics, Mannheim, Germany) treatment were incubated in Fc block (0.5 mg/mL; BD Pharmingen) for 15 minutes on ice, stained with antibodies against F4/80 (1:30; Serotec) or Gr-1 ( ) (1:200; Santa Cruz Biotechnology, San Diego, Calif.), fixed in 2% PFA, and subjected to FACS analysis (FACScalibur, BD Biosciences). F4/80 or Gr-1 expression on live cells was detected by gating on forward scatter versus side scatter characteristics, followed by analysis of F4/80 and Gr-1 in the fluorescence channels.

### Statistics

### Volume of CNV

Because the probability of each laser lesion developing CNV is influenced by the group to which it belongs, the mouse, the eye, and the laser spot, the mean lesion volumes were compared using a linear mixed model with a split plot repeated measures design, as previously described (Sakurai, et al., (2003), Invest Opthalmol Vis Sci 44, 2743-2749; Sakurai et al., (2003), Invest Opthalmol Vis Sci 44, 3578.3585). The whole plot factor was the genetic group to which the animal belonged while the split plot factor was the eye. Statistical significance was determined at the 0.05 level. Post hoc comparison of means was constructed with a Bonferroni adjustment for multiple comparisons.

### Protein Levels

VEGF protein data are represented as the mean ±s.e.m. of at least 3 independent experiments and compared using a two-tailed Student's t-test. The null hypothesis was rejected at P<0.05.

### Example 1

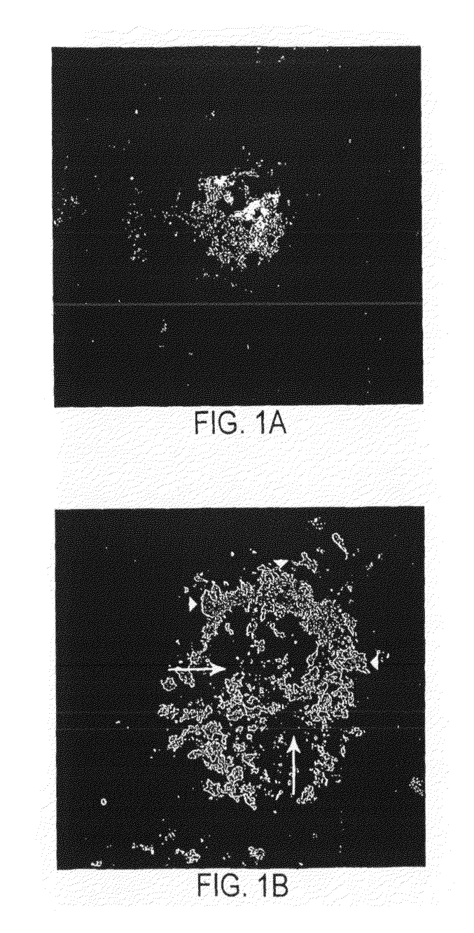

In wild-type mice, macrophages invaded the site of laser injury within 1 day, with a peak response at three days, followed by rapid disappearance by five to seven days, temporally paralleling the synthesis of MCP-1 and CCL3 mRNA, which peaked at one day after laser photocoagulation and declined nearly to undetectable pre-injury levels (data not shown) by three days (FIG. 1). MCP-1 and CCL3 expression and macrophage infiltration were restricted to the site of injury and not generalized throughout the eye (data not shown). The maximal amount of VEGF protein detected in the laser lesion at three days was significantly lower in MCP-1^{-/-}, CCL3^{-/-}, CCR2^{-/-}, or CCR5^{-/-} mice than in both control groups (FIG. 1d) and correlated with the volume of CNV at 14 days (r²=0.987).

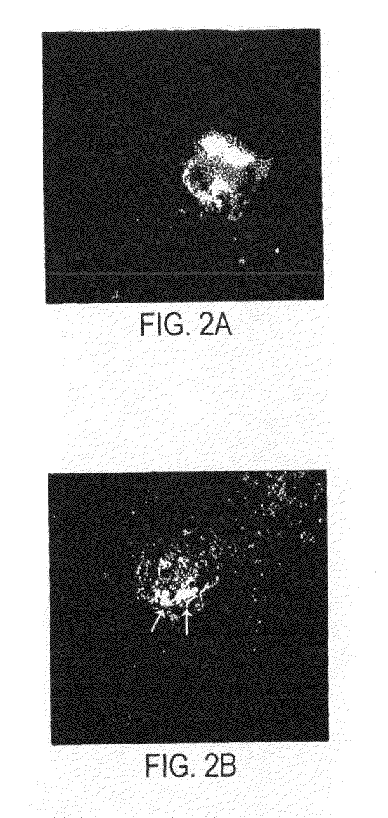

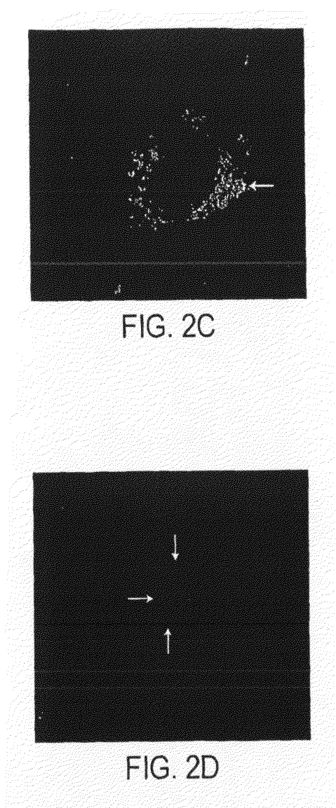

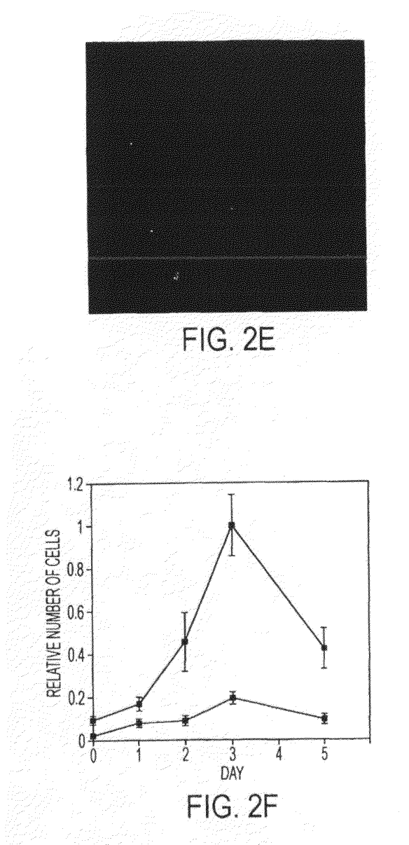

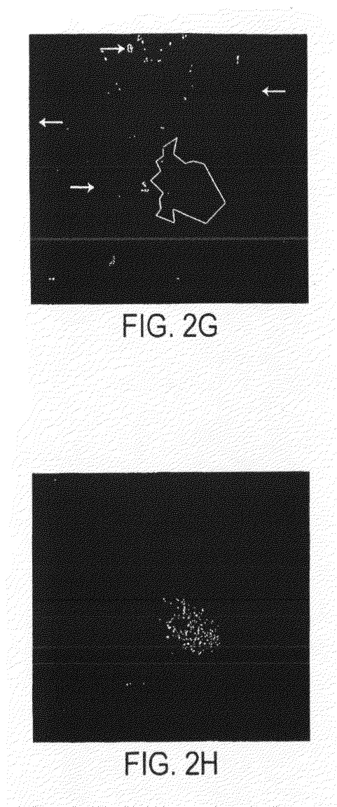

CCR2 positive cells appeared to enter the site of laser injury from the adjacent choroid within one day (FIG. 2a). Infiltration of CCR2 positive cells peaked three days after injury and declined to near-baseline levels by day 5 (FIG. 2b,c). On days one and two, the spatial pattern of F4/80 staining was nearly identical to CCR2 staining, whereas on days three and five, several areas of F4/80 staining were devoid of CCR2 staining (FIG. 2c-e), suggesting that the early macrophage response consists predominantly of CCR2 expressing cells, whereas other monocyte subsets may contribute to the later response. Flow cytometry studies confirmed that the vast majority of resident F4/80 positive cells (88.5±3.8%) in the pre-injury choroid did not express CCR2, whereas on day one, a large fraction of F4/80 positive cells (46.8±9.4%) also expressed CCR2 (FIG. 2f), suggesting that recruited, and not resident, macrophages account for the major contribution to CNV. On ensuing days only a minority of F4/80 positive cells also expressed CCR2.

Although CCR2 expression was originally described on monocyte derived cells, endothelial cells recently have been described to express CCR2 (Weber, et al., (1999), Arterioscler Thromb Vasc Biol 19, 2085-2093; Salcedo, et al., (2000), Blood 96, 3440). Prior to laser injury most CCR2 positive cells in the choroid (93.0±2.3%) did not express F4/80, indicating that at least a subset of endothelial cells in the choroid express CCR2. After laser injury, the number of CCR2 positive cells increased monotonically for three days and then declined precipitously, paralleling the fall in number of F4/80 positive cells. Combined with the observation that endothelial staining, both by CD31 or lectin, increases monotonically for at least two weeks, we can conclude that the majority of CCR2 positive cells in CNV are macrophages and not endothelial cells.

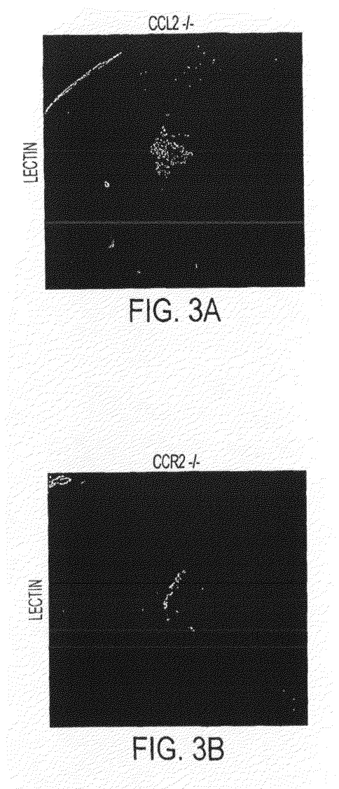

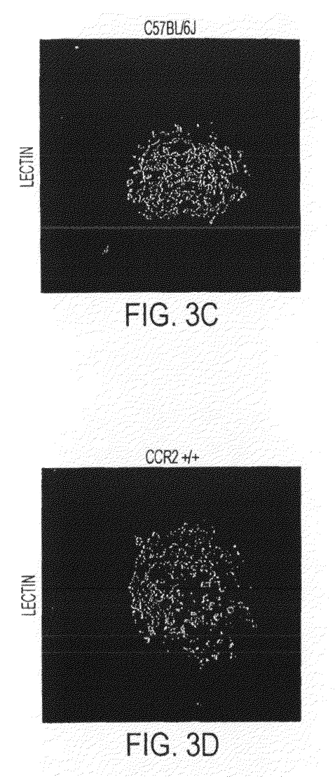

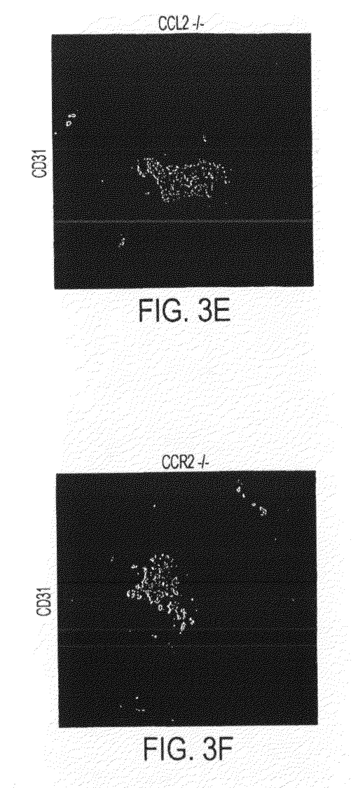

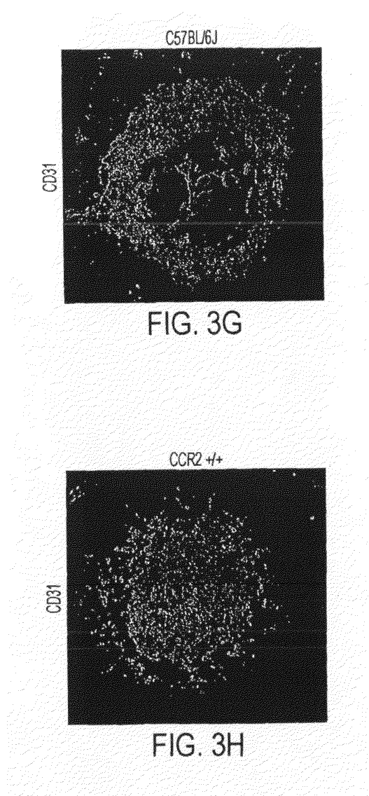

Flow cytometry studies showed that the peak macrophage response, which occurs at three days after injury, was markedly attenuated both in CCL3^{-/-} and CCR5^{-/-} mice compared with controls (FIG. 3a). Although CCL3 can chemoattract neutrophils, the neutrophil response, which peaks at 1 day after laser injury (data not shown), was not different between controls and knockouts (FIG. 3b).

### Example 2

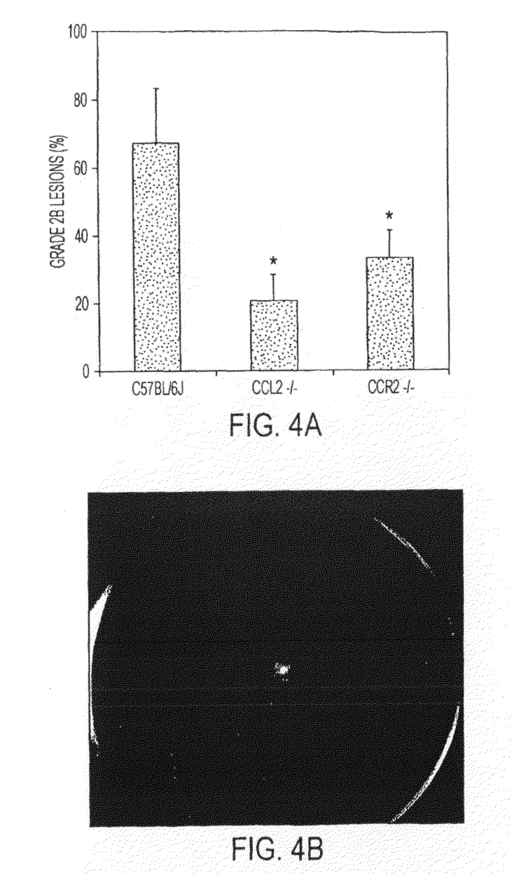

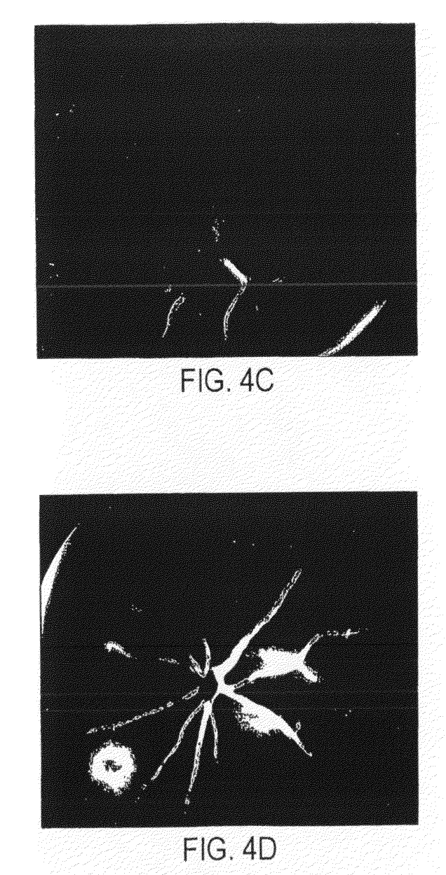

No evidence of a significant contribution of resident retinal macrophages was detected in experiments with pharmacological depletion. Staining with 5D4, which identifies an epitope on microglia not found on blood-borne monocytes and macrophages, indicated that there is not a significant microglial presence in CNV on the first three days after laser injury (FIG. 2g,h). Cells stained by 5D4 were observed in the neural retina, but not in the substance of the CNV lesion, nor were they differentially distributed at the retinal edges of the lesion. The volume of CNV in MCP-1^{-/-} mice was reduced by greater than 75% (P<0.0001), and by greater than 70% (P<0.0001) in CCR2^{-/-} mice, compared to wild-type mice at two weeks following laser injury (FIG. 3), both by lectin and CD31 staining. Because lectin has been reported to stain not only endothelial cells but also microglia, volume measurements were confirmed using CD31 staining. The volumes obtained by the two methods of staining were highly correlated (r²=0.975). There was no statistically significant difference in CNV volume between the two knockout strains as measured by CD31 (P=0.60) or lectin (P=0.39) staining, or between eyes within genetically identical groups (P>0.20 for all groups by both staining methods). To correct for potential genetic drift among C57BL/6J mice, littermates of CCR2^{-/-} mice were used as a second control. There was no statistically significant difference in CNV volume between the two control groups as measured by CD31 (P=0.64) or lectin (P=0.69) staining. At the same time point, most lesions in wild-type mice exhibited pathologically significant fluorescein leakage, whereas most lesions in MCP-1^{-/-} and CCR2^{-/-} mice did not (FIG. 4). The volume of CNV in CCL3^{-/-} mice was reduced by greater than 75% (P<0.0001), and by greater than 70% (P<0.0001) in CCR5^{-/-} mice, compared to wild-type mice at two weeks following laser injury (FIG. 4).

### Example 3

This example shows that neutralization of MCP-1 by means of anti-MCP-1 antibody significantly inhibited macrophage recruitment and resulted in a concomitant reduction of CNV by about 77%.

Neutralizing hamster antibody specific for mouse MCP-1 (1 ng; BD Biosciences, 2350 Qume Drive San Jose, Calif.), VEGF-A₁₆₄ (4 pg-4 ng), mouse MCP-1 (0.3-1 ng), neutralizing goat antibodies against mouse VEGF-A (102 ng), mouse VEGFR-1 (1-6 μg), and mouse VEGFR-2 (25-250 ng) were dissolved in phosphate buffered saline (PBS). The various antibodies were injected into the vitreous cavity of mice (wild-type, Mcp-1^{-/-}, Vegfr 1 tk^{-/-} mice) whose eyes had been laser photocoagulated to induce CNV as described above in a total volume of 1 μl.

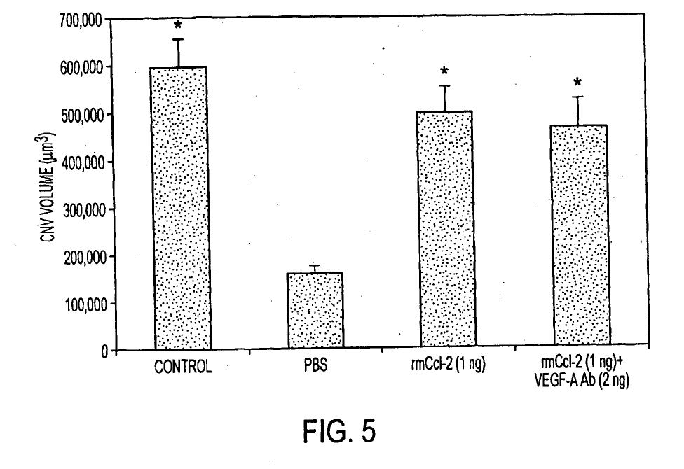

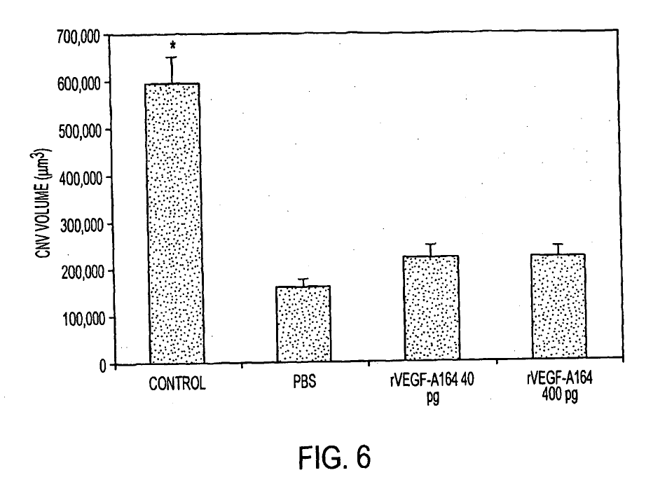

Recombinant MCP-1 (0.55 ng) restored the CNV inhibited by anti-VEGF-A antibody to 95.7±13.0% of control (n=6, P=0.82), confirming that downstream suppression of MCP-1 is the proximate cause of the anti-angiogenic effect resulting from VEGF-A neutralization. CNV reduction in wild-type mice treated with anti-MCP-1 antibody (77±4%) and in PBS-treated MCP-1^{-/-} mice (74±3%), both outstripped CNV inhibition by anti-VEGF-A antibody (48±15%) in wild-type mice (n=8-12, P<0.05), consistent with the incomplete suppression of MCP-1 by VEGF-A antibody. Recombinant VEGF-A (4-400 pg) could not increase CNV either in wild-type mice treated with anti-MCP-1 antibody (77±4%) or in PBS-treated MCP-1 mice (FIGS. 5 and 6). Collectively these data indicate that the level of VEGF-A induced by laser injury supports angiogenesis indirectly via stimulation of MCP-1 rather than by directly recruiting macrophages.

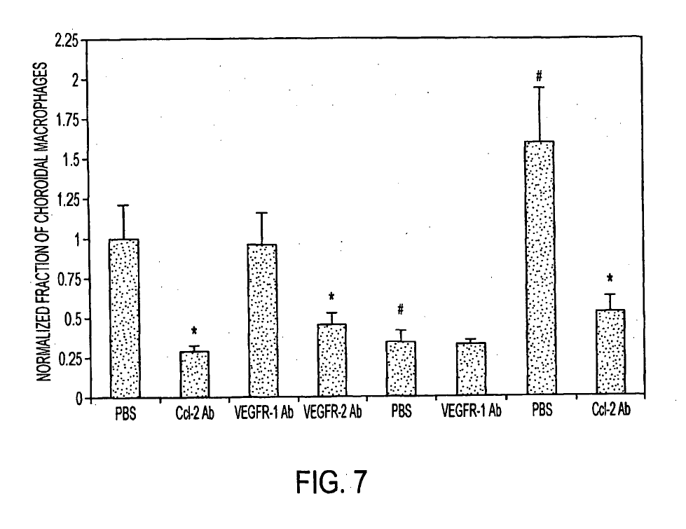

The data in FIG. 7 show that neutralization of MCP-1 in wild-type mice inhibited macrophage recruitment to the same extent as seen in MCP-1^{-/-} mice. Macrophage recruitment, which was augmented in Vegfr tk −/− mice consistent with their increased CNV (data not shown), was reduced by anti-MCP-1 antibody.

Collectively, these data show that macrophage recruitment in the eye after injury can be prevented or inhibited by administration of an anti-MCP-1 antibody to the eye.

What is claimed is:

1. A composition for treatment or prevention of ocular neovascularization comprising an effective amount of a MCP-1 antagonist and a CCR2 antagonist that blocks MCP-1 binding to or activation of CCR2.

2. The composition of claim 1 wherein the MCP-1 antagonist is an anti-MCP-1 antibody or active fragment thereof and/or the CCR2 antagonist is an anti-CCR2 antibody or active fragment thereof.

3. The composition of claim 1 further comprising an anti-inflammatory agent or steroid drug.

4. The composition of claim 1 further comprising a VEGF antagonist.

5. A method of treating or preventing ocular neovascularization comprising administering to a patient in need thereof a composition consisting essentially of an effective amount of a MCP-1 antagonist and/or a CCR2 antagonist that blocks MCP-1 binding to or activation of CCR2 and optionally, an effective amount of an anti-inflammatory agent, a steroid drug or a combination thereof.

6. The method of claim 5 wherein the composition is administered systemically.

7. The method of claim 5 wherein the composition is applied topically to the eye.

8. The method of claim 5 wherein the composition is administered by direct injection into the eye.

9. The method of claim 5 wherein the composition is administered by transscleral delivery via passive diffusion, osmotic pump, iontophoresis, ocular implant or a controlled release device.

10. The method of claim 5 wherein the MCP-1 antagonist is an anti-MCP-1 antibody or active fragment thereof.

11. The method of claim 5 wherein the CCR2 antagonist is an anti-CCR2 antibody or active fragment thereof.

12. The method of claim 5 wherein the composition consists essentially of a MCP-1 antagonist and optionally, an anti-inflammatory agent, a steroid drug, a VEGF antagonist, or a combination thereof.

13. The method of claim 12 wherein the MCP-1 antagonist is an anti-MCP-1 antibody or active fragment thereof.

14. The method of claim 12 wherein the MCP-1 antagonist is a small molecule that interferes with MCP-1 binding to or activation of CCR2.

15. The method of claim 12 wherein the MCP-1 antagonist is an RNA/DNA aptamer.

16. The method of claim 12 wherein the MCP-1 antagonist is an siRNA.

17. The method of claim 12 wherein the MCP-1 antagonist is an MCP-1 derivative.

18. The method of claim 5 wherein the composition consists essentially of a CCR2 antagonist.

19. The method of claim 18 wherein the CCR2 antagonist is an anti-CCR2 antibody or active fragment thereof.

20. The method of claim 18 wherein the CCR2 antagonist is a small molecule that blocks MCP-1 binding to or activation of CCR2.

21. The method of claim 18 wherein the CCR2 antagonist is an RNA/DNA aptamer.

22. The method of claim 1 wherein the CCR2 antagonist is an siRNA.

23. The method of claim 5 wherein the ocular neovascularization is choroidal neovascularization associated with age-related macular degeneration.

24. The method of claim 13 wherein the ocular neovascularization is choroidal neovascularization associated with age-related macular degeneration.

25. The method of claim 24 wherein the composition consists essentially of an anti-MCP-1 antibody or active fragment thereof.

26. The method of claim 5 wherein the ocular neovascularization is choroidal neovascularization.

27. The method of claim 26 wherein the composition consists essentially of an anti-MCP-1 antibody or active fragment thereof.

28. A method of treating or preventing choroidal neovascularization associated with age related macular degeneration (AMD) comprising administering to a patient a composition consisting essentially of an MCP-1 antagonist or CCR2 antagonist, or a combination.

29. The method of claim 28 wherein the composition is administered systemically.

30. The method of claim 28 wherein the composition is applied topically to an affected eye.

31. The method of claim 28 wherein the composition is administered by direct injection into an affected eye.

32. The method of claim 28 wherein the composition is administered by transscleral delivery via passive diffusion, osmotic pump, iontophoresis, ocular implant or a controlled release device.

33. The method of claim 28 wherein the MCP-1 inhibitor is an anti-MCP-1 antibody.

34. The method of claim 28 wherein the CCR2 antagonist is an anti-CCR2 antibody.

35. The method of claim 28 wherein the composition consists essentially of a MCP-1 antagonist.

36. The method of claim 28 wherein the MCP-1 antagonist is an MCP-1 antibody.

37. The method of claim 28 wherein the MCP-1 antagonist is an RNA/DNA aptamer.

38. The method of claim 28 wherein the MCP-1 antagonist is a small molecule that interferes with MCP-1 binding to or activation of CCR2.

39. The method of claim 28 wherein the MCP-1 antagonist is an siRNA.

40. The method of claim 28 wherein the composition consists essentially of a CCR2 antagonist.

41. The method of claim 40 wherein the CCR2 antagonist is an antibody.

42. The method of claim 40 wherein the CCR2 antagonist is an RNA/DNA aptamer.

43. The method of claim 40 wherein the CCR2 antagonist is a small molecule that interferes with MCP-1 binding to or activation of CCR2.

44. The method of claim 39 wherein the CCR2 antagonist is an siRNA.
